(19) United States
(12) Patent Application Publication
(10) Pub. No.: US 2005/0137663 A1
(43) Pub. Date: Jun. 23, 2005

(54) Headache relief band

(76) Inventors: Tanya Lopez, Sarasota, FL (US); Jon P. Capriola, Sarasota, FL (US)

Correspondence Address: Richard C. Litman;LITMAN LAW OFFICES, LTD., P.O. Box 15035, Arlington, VA, 22215, US

(21) Appl. No.: 10/739,039

(22) Filed: Dec. 19, 2003

(51) Int. Cl. A61F 7/00

(52) U.S. Cl. 607/110

(57) ABSTRACT

A headband for relieving the pain of a headache. The headband is fabricated from a single strip of elastic material. The elastic material is covered with a moisture-absorbing fabric. The fabric may be made in different colors to satisfy a user's aesthetic taste. Hook and loop fasteners are secured to the ends of the single strip for ease in attaching the band to the head and for adjusting the tension. The band can be misted with water and frozen for cool relief.

### BACKGROUND OF THE INVENTION

1. Field of the Invention

The present invention generally relates to pain relief devices. More specifically, the present invention is drawn to a band adapted for wear on a user's head to relieve headache pain.

2. Description of the Related Art

The common headache is probably the most universally occurring malady to afflict the human race. At any given moment literally millions and millions of people on planet earth will have a headache. Oral medications have proven to be effective to relieve the headache pain. Some people, however, are reluctant to use oral medications because of adverse side effects or the like. An inexpensive, non-oral, easy-to-use headache remedy would certainly be a welcome addition to the art.

The related art is rife with non-oral headache relieving devices. For example U.S. Pat. No. 6,379,295 B1 (Woo) and Japanese Patent numbered JP10015084 (Iasmu et al.) disclose the use of magnets for relieving headache pain.

U.S. Pat. No. 4,781,193 (Pagden) shows a headache treatment apparatus that simultaneously heats and cools different portions of a wearer's head.

U.S. Pat. No. 6,083,186 (Zajac) shows a headband assembly that comprises a flat plastic substrate, copper-electrical wire and aluminum foil.

U.S. Pat. No. 5,700,238 (Hyson) is drawn to a device that includes an opaque segment for covering the eyes.

U.S. Pat. No. 5,338,290 (Aboud) employs a band made from multiple strips of elastic stacked on top of one another.

U.S. Pat. No. 5,419,758 (Vijayan) utilizes rubber discs adapted for insertion under an elastic band.

U.S. Pat. No. 5,848,981 (Herbranson) discloses an elongate band that includes compressive cooling material for engagement with a wearer's head.

U.S. Pat. No. 6,554,787 B1 (Griffin et al.) is drawn to a headband having pockets for the insertion of treatment elements.

None of the above inventions and patents, taken either singly or in combination, is seen to disclose a simplistic headache-relieving headband as will be subsequently described and claimed in the instant invention.

### SUMMARY OF THE INVENTION

The present invention is drawn 'to a headband for relieving the pain of a headache. The headband is fabricated from a single strip of elastic material. The elastic material is covered with a moisture-absorbing fabric (fleece). The fabric may be made in different colors to satisfy a user's aesthetic taste. Hook and loop fasteners are secured to the strip for ease in attaching the band to the head and for adjusting the tension. The band can be misted with water and frozen for cool relief. The instant invention is effective to control migraine, tension cluster, hangovers and rebound headaches.

Accordingly, it is a principal object of the invention to provide a device, which device is effective to relieve headache pain.

It is another object of the invention to provide a device for relieving headache pain, which device can be worn on a user's head.

It is a further object of the invention to provide a device for relieving headache pain, which device is comfortable to wear.

Still another object of the invention is to provide a device for relieving headache pain, which device is easy to use.

It is an object of the invention to provide improved elements and arrangements thereof for the purposes described which are inexpensive, dependable and fully effective in accomplishing their intended purposes.

These and other objects of the present invention will become readily apparent upon further review of the following specification and drawings.

### BRIEF DESCRIPTION OF THE DRAWINGS

FIG. 1 is an environmental, perspective view of a headache relief band according to the present invention.

FIG. 2 is a plan view of a headache relief band according to the present invention.

Similar reference characters denote corresponding features consistently throughout the attached drawings.

### DETAILED DESCRIPTION OF THE PREFERRED EMBODIMENT

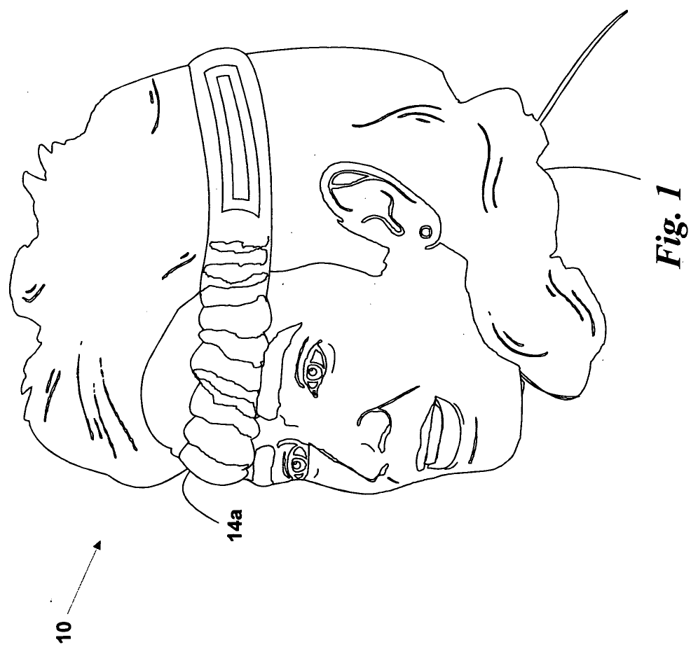

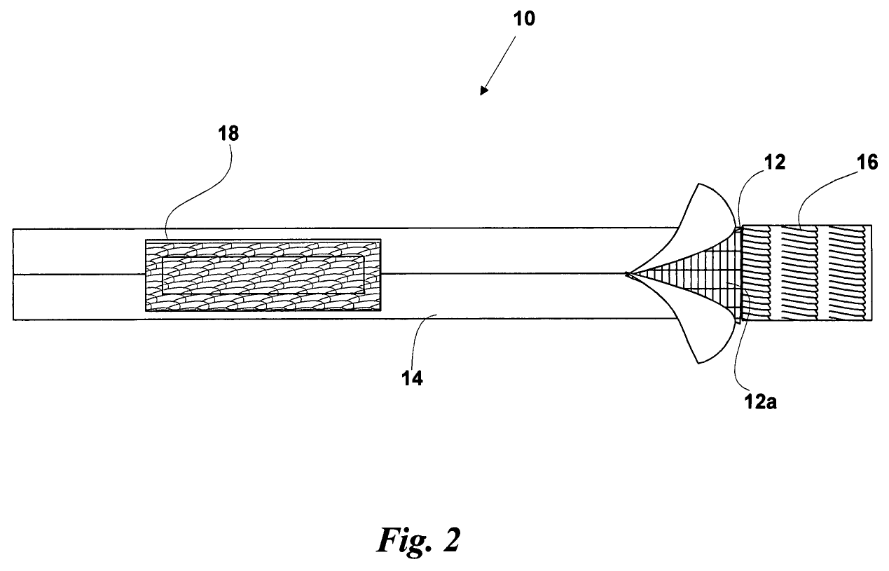

The headache relief band of the present invention is generally indicated at 10 as illustrated in FIGS. 1 and 2. Band 10 incorporates a substrate that comprises a single strip of durable elastic material 12. Elastic strip 12 is covered with a soft, moisture-absorbing fabric 14. In a relaxed state the band has a length of approximately twenty-two inches. The elastic substrate permits the band to be stretched to approximately forty inches. The moisture-absorbing fabric is gathered at 14a to allow the fabric to stretch with the underlying elastic strip. At least the inner surface 12a of the strip is a smooth planar surface, unencumbered by any surface irregularities. A hook and loop fastener 16, is attached to and extends from one end of strip 12. A second hook and loop fastener 18 is disposed on the rear surface of fabric 14. This arrangement allows the band to be easily positioned and adjusted (tight or loose) on a wearer's head for a comfortable fit. The band can be reused many times whenever a headache develops. Relief normally occurs within twenty-thirty minutes. As noted above, the band may be misted with water and frozen or heated with hot water if cool or warm relief is desired.

It is to be understood that the present invention is not limited to the embodiment described above, but encompasses any and all embodiments within the scope of the following claims.

1. A band adapted to be worn on the head for headache relief, comprising:
an elastic strip, said strip having a uniform width, a first end, a second end and at least one smooth planar surface;
a covering material encompassing said elastic strip, said covering material fabricated from a soft, pliable fabric;
a first fastener attached at and extending from said first end and a second fastener attached to said covering material between said first end and said second end, whereby said band can be easily secured to a user's head.

2. The band as recited in claim 1, wherein said covering material is a moisture-absorbing fabric.

3. The band as recited in claim 1, wherein said first fastener is a hook and loop fastener.

4. The band as recited in claim 1, wherein said second fastener is a hook and loop fastener.

5. The band as recited in claim 1, wherein said at least one surface is unencumbered by any surface irregularities.

6. A band adapted to be worn on the head for headache relief, comprising:
an elastic strip, said strip having a uniform width, a first end, a second end and at least one smooth planar surface, said surface being unencumbered by any surface irregularities;
a covering material encompassing said elastic strip, said covering material fabricated from a soft, pliable, moisture-absorbing fabric;
a first fastener attached at and extending, from said first end and a second fastener attached to said covering material between said first end and said second end, whereby said band can be easily secured to a user's head.

7. The band as recited in claim 6, wherein said first fastener and said second fastener are hook and loop fasteners.
